(19) United States
(12) Patent Application Publication
(10) Pub. No.: US 2009/0076608 A1
(43) Pub. Date: Mar. 19, 2009

(54) Intervertebral disc replacement prosthesis

(75) Inventors: Jeffrey David Gordon, Saratoga Springs, NY (US); John K. Song, Chicago, IL (US)

Correspondence Address: JOHN K. SONG, 474 NORTH LAKE SHORE DR., APT. # 4508, CHICAGO, IL, 60611-6465, US

(73) Assignee: Vermillion Technologies, LLC, Chicago, IL (US)

(21) Appl. No.: 11/901,389

(22) Filed: Sep. 17, 2007

(51) Int. Cl. A61F 2/44; A61B 17/56

(52) U.S. Cl. 623/17.16; 623/17.15; 623/17.11

(57) ABSTRACT

An intervertebral disc replacement prosthesis for placement between a first vertebra and a second vertebra adjacent to the first vertebra. In one embodiment, the intervertebral disc replacement prosthesis includes a resilient member, which is formed from a single flat member, and three support members. The support members are housed in the resilient member that is arranged, in use, to be secured to the first and second vertebrae. The intervertebral disc prosthesis can generate a coupled motion in more than one possible direction responsive to a possible movement of at least one of the first and second vertebrae, among the resilient member, the first support member, the second support member, and the third support member.

### FIELD OF THE INVENTION

The present invention generally relates to a device for treatment of spine disorders, and in particular to the utilization of an intervertebral disc prosthesis to perform one or more functions of an intervertebral disc between an adjacent pair of vertebrae.

### BACKGROUND OF THE INVENTION

Degenerative spinal disease results from the progressive degeneration of the spinal disc (common terms are “disc bulge”, “slipped disc”, “herniated disc”) and of the articulations between the bones of the spine called the facets (resulting in painful and enlarged joints). It is a major source of disability and lost work. Symptoms of the disease are back pain caused by painful joint contact, overstrained ligaments and muscles, and numerous other factors. In addition, nerve pain (“radiculopathy”, “sciatica”) can occur from pressure on nerves from bone spurs, herniated discs, and/or narrowing of the foramen (the nerve root path through the spine).

Current treatment of degenerative spine disease can range from non-surgical methods such as physical therapy, pain medication and rest, to removal of bone spurs and/or herniated discs. Increasingly more prevalent is the use of spinal fusion whereby screws and rods are used to fix the painful joints in place. However, it is becoming recognized that while this works in the short term, in the long term the adjacent disc levels are forced to over-extend because of the loss of motion at the fused level. Subsequently, these adjacent levels degenerate more quickly and often require additional surgery.

Total Disc Arthoplasty (TDA) is the newest and most advanced area of research in spinal surgery. TDA is the replacement of the spinal disc and is intended to relieve pain while maintaining normal spinal motion to prevent adjacent discs from degenerating. In Europe, TDA has been practiced for over 20 years. A recognized benefit has been dramatically shortened patient recovery periods as compared to fusion.

As TDA is a novel technology, it must gain acceptance within the medical community. In order to do so, clinical efficacy, safety and patient satisfaction must be demonstrated. In addition, surgeon comfort with the implant and implantation procedure must be established. There are a handful of disc replacement devices in development and in clinical trials. However, examination of current technology has revealed that the devices under development do not correctly or adequately address a multitude of factors including: re-establishment of normal spinal motion, durability, simplicity, and ease of use.

### SUMMARY OF THE INVENTION

The preferred embodiment of the present invention is an intervertebral disc replacement prosthesis for placement between two adjacent vertebrae which comprises a resilient member which contacts both vertebrae and three support members. The resilient member is created by cutting a shape into sheet metal and then bending or otherwise deforming said cut piece. The final form of the resilient member consists of two plates for contacting each of the adjacent vertebrae, a flexure strip, and the cavity produced by these three features. In one embodiment, three support members are housed within the cavity of the resilient member: (1) a first support member having a bearing surface, a non-bearing surface and a body portion therebetween; (2) a second support member also having a bearing surface, a non-bearing surface, and a body portion therebetween, and; (3) a third support member having a first bearing surface, a second bearing surface and a body portion therebetween. The arrangement of the support members is such that the third support member is sandwiched between the first and second support members with its bearing surfaces articulating with the bearing surfaces of the first and second support members. The first and second support members may be attached to the resilient member, or may have at least one degree of freedom restricted by the resilient member. Preferably, the bearing surface of the first support member is concave and articulates with a convex bearing surface of the third support member, and the bearing surface of the second support member is planar and articulates with a planar bearing surface of the third support member. Alternatively, one of these planar bearing surfaces may include at least one boss which fits within a cavity in the opposite planar bearing surface thereby limiting the extent of translation possible by the planar articulation. The convex/concave articulation could have such a boss/cavity feature to limit motion in addition or inlieu of the planar articulation boss/cavity feature. The stiffness inherent to the flexure strip of the resilient member provides significant resistance to any rotational and/or planar forces exerted on the device, similar to a natural intervertebral disc.

According to another embodiment of the invention, two support members are housed within the cavity of the resilient member: (1) a first support member having a bearing surface, a non-bearing surface and a body portion therebetween; (2) a second support member also having a bearing surface, a non-bearing surface, and a body portion therebetween. The arrangement of the support members is such that the bearing surfaces of the first and second support members cooperate with each other. Also, the first and second support members may be attached to the resilient member, or may have at least one degree of freedom restricted by the resilient member. Preferably, the bearing surface of the first support member is concave and articulates with a convex bearing surface of the second support member. Similar to the above boss/cavity features described above, this embodiment could also incorporate a boss/cavity feature to limit articulation motion. The stiffness inherent to the flexure strip of the resilient member provides significant resistance to any rotational forces exerted on the device.

The present invention may incorporate a number of different means of attachment to the adjacent vertebrae. In one embodiment, partial cylinder bosses are present on the two endplates of the device. These bosses are roughly concentric so as to conform with a feature cut into the vertebrae with a rotating drill or reamer. This feature can be used to prevent translational migration of the implant in several planes and rotational migration about several axes, and may provide an area of tight implant/bone contact. Additionally, tabs incorporated into the device may allow for bone screws to be utilized to fix the implant to the vertebrae. Also, teeth incorporated into the endplates which bite into the vertebrae could be used to fix the implant to the vertebrae

It is an object of the present invention to provide an apparatus for placement between two adjacent vertebrae which acts to mimic the motion of a healthy intervertebral disc.

It is another object of the present invention to provide an apparatus for placement between two adjacent vertebrae which acts to mimic the stiffness of a normal intervertebral disc.

It is another object of the present invention to provide an apparatus for placement between two adjacent vertebrae which is substantially contained and connected so as to be a single unit. The advantages of such an apparatus include ease of surgical placement of the apparatus and prevention of migration of one or more portions of the apparatus from the surgically implanted site.

### BRIEF DESCRIPTION OF THE DRAWINGS

FIG. 1A shows the sheet metal cutout used to create one embodiment of the invention

FIG. 1B shows the same sheet metal cutout shown in FIG. 1A after some bending

FIG. 1C shows the same sheet metal cutout shown in FIG. 1B after further bending and after insertion of bearing pieces

FIG. 1D shows the same sheet metal cutout shown in FIG. 1C after final bending

FIG. 2 shows the invention with screws inserted

FIG. 3 shows the invention in an extended configuration

FIG. 4 shows the invention in a flexed configuration.

FIG. 5A is an exploded view of the first, second and third support members according to one embodiment of the invention

FIG. 5B is an exploded view of the first, second and third support members according to one embodiment of the invention from a view reverse to FIG. 5A

FIG. 6A is an exploded view of the first and second support members according to an alternative embodiment of the invention

FIG. 6B is an exploded view of the first and second support members according to an alternative embodiment of the invention from a view reverse to FIG. 6A

FIG. 7A is a front view of one embodiment of the invention

FIG. 7B is a cross-sectional view of the embodiment of the invention shown in FIG. 7A

FIG. 8A is a front view of an another embodiment of the invention

FIG. 8B is a cross-sectional view of the embodiment of the invention shown in FIG. 8A

FIG. 9A is a perspective view of another embodiment of the invention shown prior to a final bend

FIG. 9B is a perspective view of the embodiment of the invention shown in FIG. 9A after final bend

FIG. 10 is a front view of the embodiment of the invention shown in FIGS. 9A and 9B

FIG. 11A is a perspective view of another embodiment of the invention

FIG. 11B is a perspective view of the embodiment of the invention shown in FIG. 11A prior to a final bend

FIG. 12A is an exploded perspective view of support members used in one embodiment of the invention

FIG. 12B is an exploded perspective view of support members shown in FIG. 12A from a reverse angle

FIG. 13 is a cross sectional view of the support members shown in FIGS. 12A and 12B

### DETAILED DESCRIPTION OF THE DRAWINGS

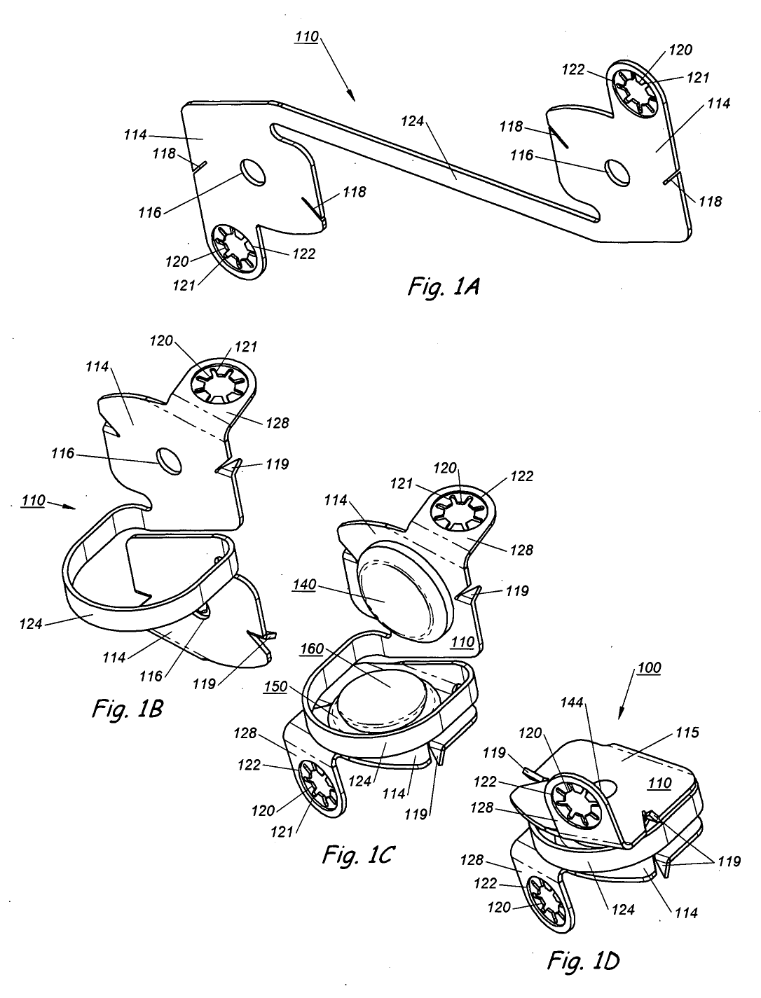

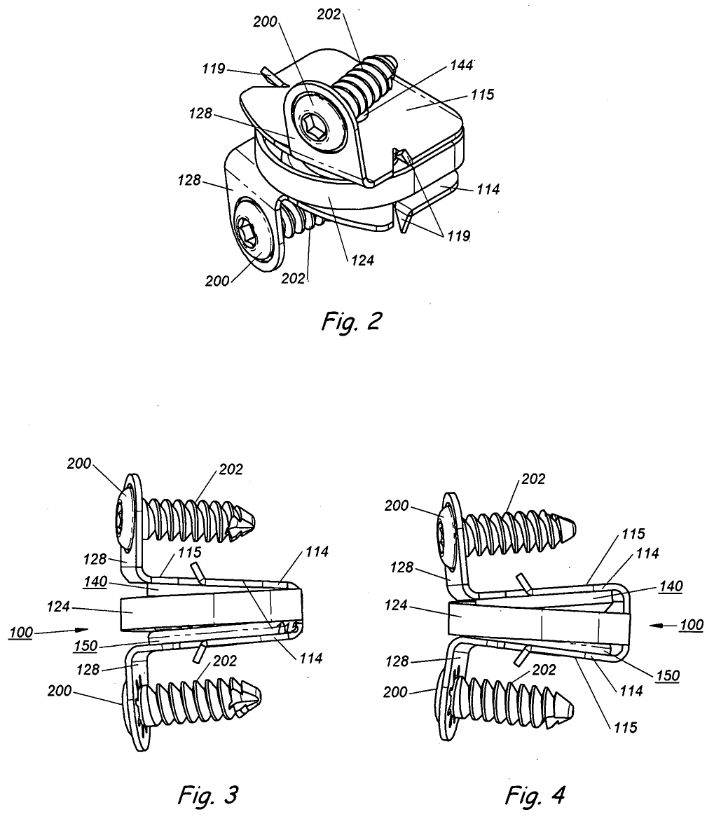

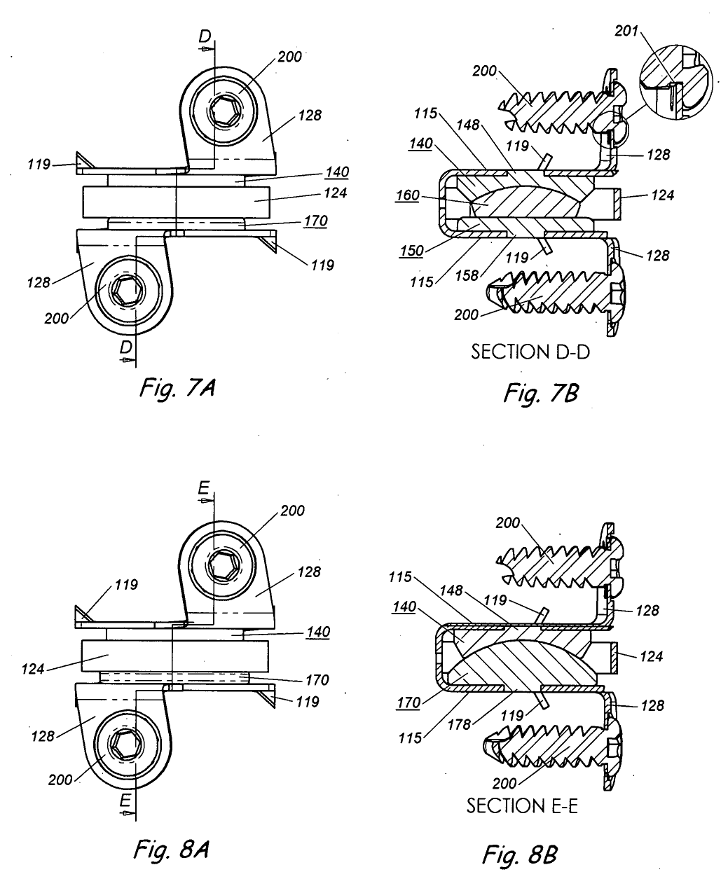

FIGS. 1A through 1D show a suggested procedure for creating a resilient member 110. The blank is cut or punched from a thin sheet of a biocompatible, implantable material. The blank may have many different shapes but in general it may include endplates 114 for contacting the vertebrae and a flexure strip 124 which gives the device flexibility and may additionally incorporate tabs 128 to prevent migration of the implant 100 too far into the disc space. A bend is made in flexure strip 124 so that resilient member 110 appears as in FIG. 1B. Also, tabs 128 and teeth 119 have been created by bending appropriate portions of endplates 114. For example, slits 118 facilitate the creation of teeth 119. One endplate 114 is bent and first support member 140 and second support member 150 are attached to endplates 114. Third support member 160 is positioned so that the implant 100 looks like FIG. 1C. A final bend completes the assembly so that implant 100 looks like FIG. 1D. Tabs 128 may incorporate holes 120 to accommodate screws 200. Shown in the figures is a screw capturing mechanism consisting of a hole 120, slits 121 and a counterbore 122. FIG. 2 shows placement of screws into holes 120. Slits 121 in counterbore 122 create thin tabs 123. Screw threads 202 have a larger major diameter than the diameter of hole 120, and screw shoulder 201 has a smaller diameter than the diameter of hole 120. Therefore, tabs 123 flex to allow passage of screw threads 202 through hole 120, then will return to their initial position after screw threads 202 have completely passed through and tabs 123 are adjacent to screw shoulder 201 (see FIG. 7B).

FIG. 3 shows the implant 100 in an extended position. Flexure strip 124 deforms in reaction to bending loads. First support member 140 rotates about third support member 160 in a ball-and-socket manner, and third support member 160 slides on second support member 150 to accommodate any translational motion generated by the extension bending. Therefore, implant 100 can accommodate motions about a number of centers of rotation. Similarly, FIG. 4 shows the implant 100 in a flexed position. Bending about axes perpendicular to the axis about which FIGS. 3 and 4 are bent is also possible by deforming at least flexure strip 124.

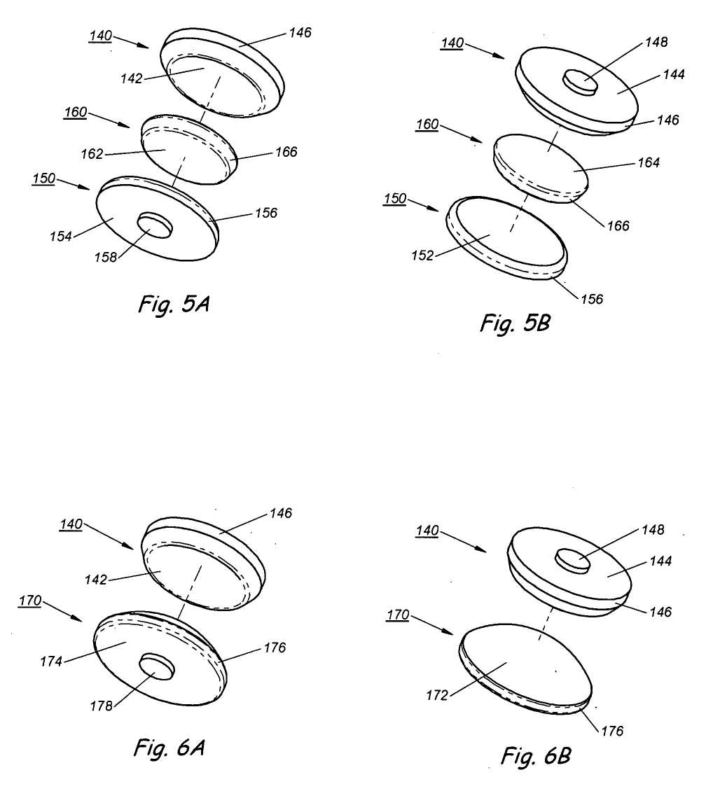

FIG. 5A shows an exploded view of first support member 140, second support member 150 and third support member 160. FIG. 5B shows the reverse angle view. First support member 140 has a bearing surface 142 which is concave, a non-bearing surface 144, and a body portion 146 therebetween. Second support member 150 has a bearing surface 152 which is substantially planar, a non-bearing surface 154, and a body portion 156 therebetween. Third support member 160 has a first bearing surface 164 which is substantially convex, a second bearing surface 162 which is substantially planar, and a body portion 166 therebetween. Concave bearing surface 142 of first support member 140 cooperates with convex first bearing surface 164 of third support member 160, and substantially planar bearing surface 152 of second support member 150 cooperates with substantially planar second bearing surface 162 of third support member 160. A boss 148 projecting from non-bearing surface 144 of first support member 140 is meant to attach first support member 140 to resilient member 110. Similarly, a boss 158 projecting from non-bearing surface 154 on second support member 150 is meant to attach second support member 150 to resilient member 110.

FIG. 6A shows an exploded view of first support member 140 and an alternative second support member 170. FIG. 6B shows the reverse angle view. Alternative second support member 170 has a bearing surface 172 which is convex, a non-bearing surface 174, and a body portion 176 therebetween. Concave bearing surface 142 of first support member 140 cooperates with convex bearing surface 172 of alternative second support member 170. A boss 178 projecting from non-bearing surface 174 of alternative second support member 170 is meant to attach alternative second support member 170 to resilient member 110.

Alternatively, bearing surface 142 of first support member 140 is convex and either second bearing surface 164 of third support member 160 or bearing surface 172 of alternative second support member 170 is concave.

FIG. 7A shows a front view of one embodiment of the invention and FIG. 7B shows a cross-sectioned view according to staggered section line DD. Cooperation of first support member 140, third support member 160 and second support member 150 is shown. Also, as stated above, the screw retaining system is shown.

FIG. 8A shows a front view of another embodiment of the invention with two support members and FIG. 8B shows a cross-sectioned view according to staggered section line EE. Cooperation of first support member 140 and alternative second support member 170 is shown.

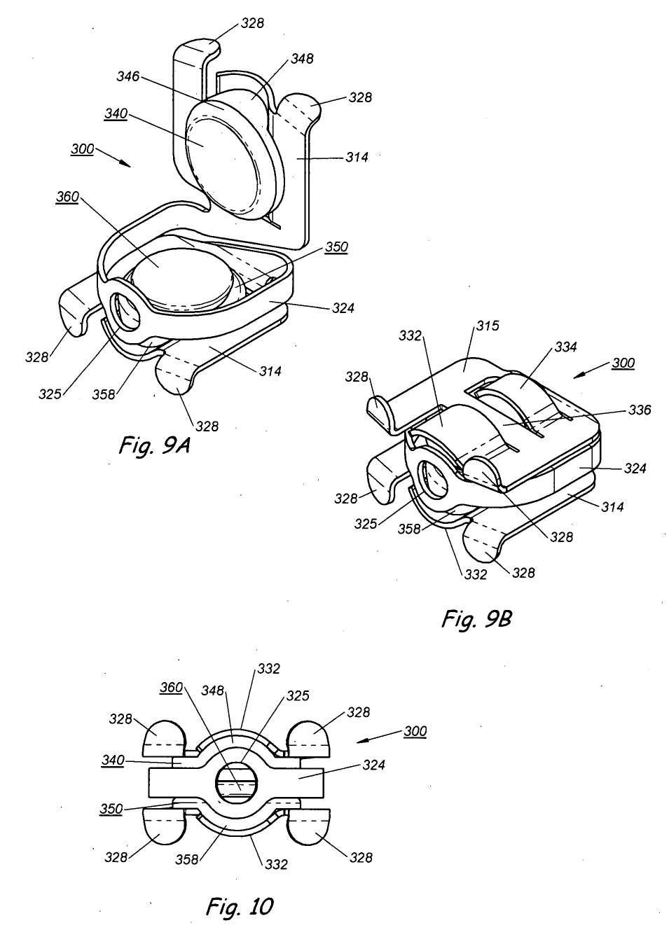

FIG. 9A shows another embodiment of the implant 300 wherein endplates 314 each have anterior tabs 332 and posterior tabs 334 which are concentric when viewed from a frontal view (FIG. 10). A boring tool (not shown) introduced into the intervertebral disc space will cut a circular shape which substantially matches the circle formed by the aforementioned tabs (see FIGS. 14A and 14B). Therefore, anterior tabs 332 and posterior tabs 334 will have close contact with the bored vertebrae, reducing implant migration and aiding bone ingrowth into a porous coating applied to anterior tabs 332 and posterior tabs 334. Also, anterior tabs 332 and posterior tabs 334 will act to prevent rotation of alternative implant embodiment 300 relative to the vertebrae to which it is attached.

FIG. 11A through 14B show another embodiment of the invention which incorporates several additional features. Partial cylinder bosses 405, similar to the ones described above, are incorporated into endplates 415. Screw tabs 428 allow the invention to be attached to the vertebrae with bone screws 200. Cut 422 allows passage of a bone screw 200 with a major diameter larger than the diameter of screw hole 421 by allowing expansion of screw hole 421. Shoulder 201 incorporated into bone screw 200 (see detail of FIG. 7B) with a diameter equal to or smaller than the unstressed diameter of screw hole 421 allows screw tab 428 to return to it's unexpanded position, thereby capturing bone screw 200.

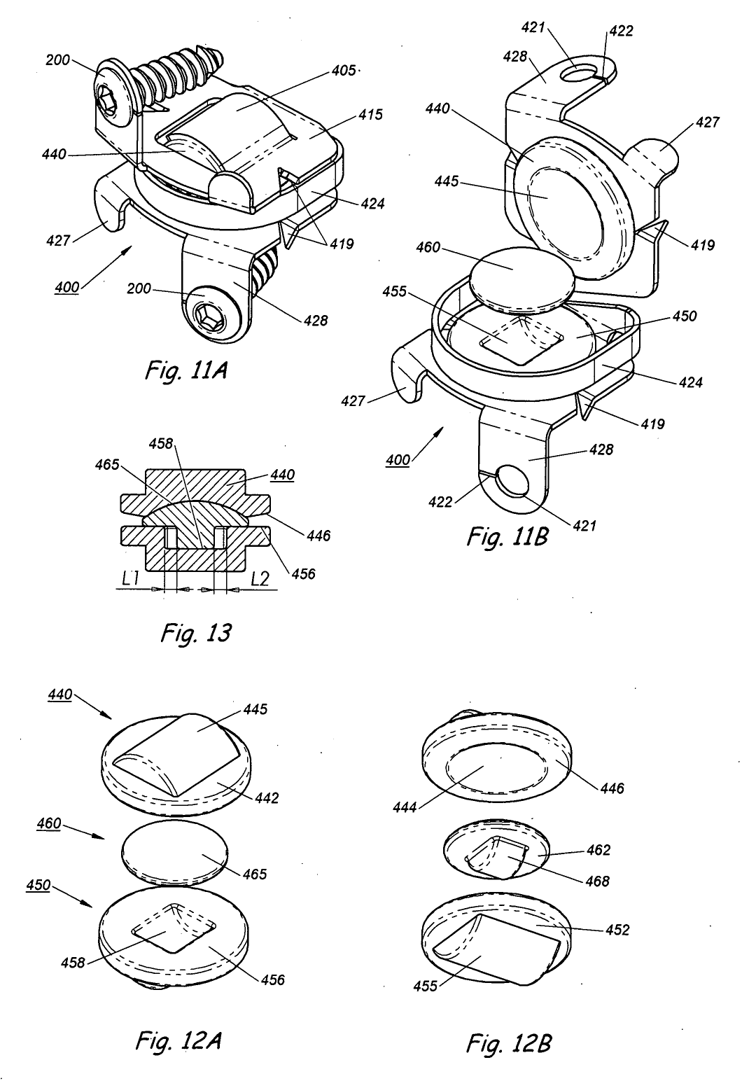

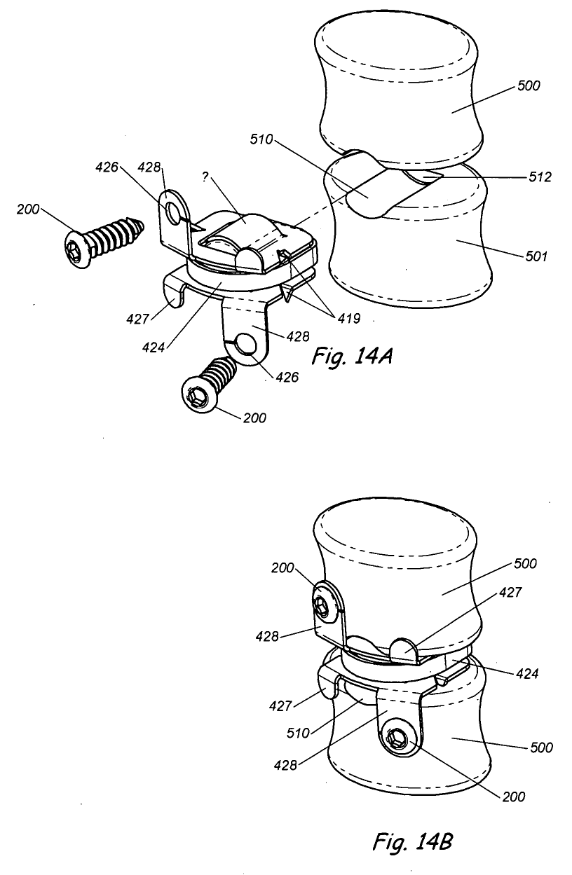

Third support member 460 incorporates a motion limiting boss 468 which slides within cavity 458 in second bearing member 450. FIGS. 12A and 12B show opposite angle views to display both features. FIG. 13 shows a cross-section of first support member 440, second support member 450 and third support member 460 showing that forward translation of magnitude L1 and backward translation of magnitude L2 is possible. Alternatively, a cavity could be incorporated into third support member and a boss incorporated into second support member.

Surface 446 of first support member 440 will contact surface 456 of second support member 450 so that rotational articulation of first member 440 with third support member 460 is limited. Partial cylinder boss 445 in first support member 440 and partial cylinder boss 455 in second support member 450 fit into similar features in resilient member 400. FIGS. 14A and 14B show vertebra 500 and adjacent vertebra 501 with a cylindrical cavity 510 created within them by a rotating tool (not shown). Partial cylinder bosses 405 fit relatively tightly into cavity 510.

All parts of the invention should be made from biocompatible materials such as metal (e.g. titanium, titanium alloy, stainless steel, cobalt-chrome alloy) ceramic (e.g. aluminum oxide, zirconium oxide), polymer (e.g. polyethylene, polyurethane, PEEK) or a composite material (e.g. carbon fiber). Parts may also incorporate coatings or surface treatments to improve wear resistance, corrosion resistance, lubricity, bone ingrowth, or to color-code the parts according to size for easy recognition. All bearing surfaces in all embodiments should preferably be polished.

All cited patents and publications referred to in this application are herein expressly incorporated by reference.

This invention thus being described, it will be obvious that the same may be varied in many ways. Such variations are not to be regarded as a departure from the spirit and scope of the present invention, and all such modifications as would be obvious to one of ordinary skill in the art are intended to be included within the scope of the following claims.

What is claimed is:

1. An intervertebral disc prosthesis for placement between a first vertebra and a second vertebra adjacent to the first vertebra, comprising:
a. a resilient member arranged, in use, to be secured to the first vertebra and the second vertebra, said resilient member created from a single flat piece of metal which is bent or otherwise formed into a shape which consists of a first bone contacting plate, a second bone contacting plate, a flexure strip, and a cavity;
b. a first support member having a bearing surface, a non-bearing surface and a body portion defined therebetween;
c. a second support member having a bearing surface, a non-bearing surface and a body portion defined therebetween;
d. a third support member having a first bearing surface, a second bearing surface, and a body portion defined therebetween;
wherein first, second and third support members are housed within said cavity of said resilient member such that the bearing surface of said first support member cooperates with the first bearing surface of said third support member, the bearing surface of said second support member cooperates with the second bearing surface of said third support member and the resilient member can flex and move in response to a possible movement of the first vertebra relative to the second vertebra.

2. The device of claim 1 further comprising means, such as protruding teeth or a means for attachment with a bone screw, to attach said intervertebral disc prosthesis to at least one vertebra.

3. The device of claim 1 further comprising means to limit migration of said intervertebral disc prosthesis into the intervertebral disc space, such as at least one tab.

4. The device of claim 1 further comprising at least one protrusion from at least one of said bone contacting plates which mates with a recess created in at least one vertebra.

5. The device of claim 1 wherein said bearing surface of said first support member is concave and is substantially complementary to said first bearing surface of said third support member which is convex.

6. The device of claim 1 wherein the bearing surface of said first support member is convex and is substantially complementary to the first bearing surface of said third support member which is concave.

7. The device of claim 1 wherein the second bearing surface of said third support member incorporates a boss which fits within a cavity in the bearing surface of said second support member to limit the range of motion between the two support members.

8. The device of claim 1 wherein the bearing surface of said second support member incorporates a boss which fits within a cavity in the second bearing surface of said third support member to limit the range of motion between the two support members.

9. The device of claim 1 which further comprises at least one screw retention means

10. The device of claim 1 wherein at least one of the bone contacting surfaces of said first bone contacting plate and said second bone contacting plate is coated with a bone ingrowth promoting substance or receives a surface modification treatment for promoting bone ingrowth such as corundum blasting.

11. The device of claim 1 wherein at least one of said first support member, said second support member, and said third support member is made of a ceramic material.

12. The device of claim 1 wherein at least one of said first support member, said second support member, and said third support member is made of a biocompatible metal.

13. The device of claim 1 wherein at least one of said first support member, said second support member, and said third support member is made of a biocompatible polymer such as polyethylene, polyurethane or PEEK.

14. An intervertebral disc prosthesis for placement between a first vertebra and a second vertebra adjacent to the first vertebra, comprising:
a. a resilient member arranged, in use, to be secured to the first vertebra and the second vertebra, said resilient member created from a single piece of flat metal which is then formed into a shape which consists of a first bone contacting plate, a second bone contacting plate, a flexure strip, and a cavity;
b. a first support member having a bearing surface, a non-bearing surface and a body portion defined therebetween;
c. a second support member having a bearing surface, a non-bearing surface and a body portion defined therebetween;
wherein first and second support members are housed within said cavity of said resilient member such that the bearing surface of the first support member cooperates with the bearing surface of the second support member and the resilient member can flex and move in response to a possible movement of the first vertebra relative to the second vertebra.

15. The device of claim 14 further comprising means to attach said intervertebral disc prosthesis to at least one vertebra, such as protruding teeth or a means for attachment with a bone screw.

16. The device of claim 14 further comprising means to limit migration of said intervertebral disc prosthesis within the intervertebral disc space, such as at least one tab.

17. The device of claim 14 further comprising at least one protrusion from at least one of said bone contacting plates which mates with a recess created in at least one vertebra.

18. The device of claim 14 wherein said bearing surface of said first support member is concave and is substantially complementary to said first bearing surface of said third support member which is convex.

19. The device of claim 14 wherein said bearing surface of said first support member is convex and is substantially complementary to said first bearing surface of said third support member which is concave.

20. The device of claim 14 which further comprises at least one screw retention means

21. The device of claim 14 wherein at least one of the bone contacting surfaces of said first bone contacting plate and said second bone contacting plate is coated with a bone ingrowth promoting substance or receives a surface modification treatment for promoting bone ingrowth such as corundum blasting.

22. The device of claim 14 wherein at least one of said first support member, said second support member, and said third support member is made of a ceramic material.

23. The device of claim 14 wherein at least one of said first support member, said second support member, and said third support member is made of a biocompatible metal.

24. The device of claim 14 wherein at least one of said first support member, said second support member, and said third support member is made of a biocompatible polymer such as polyethylene or polyurethane.
